(19) United States
(12) Patent Application Publication
(10) Pub. No.: US 2022/0183791 A1
(43) Pub. Date: Jun. 16, 2022

(54) Film Tensioning Element

(71) Applicant: Ivoclar Vivadent AG, Schaan (LI)

(72) Inventor: Markus Lichtensteiger, Montlingen (CH)

(21) Appl. No.: 17/533,189

(22) Filed: Nov. 23, 2021

(30) Foreign Application Priority Data
Dec. 10, 2020 (EP) 20213108.2

(51) Int. Cl. A61C 5/90

(52) U.S. Cl. CPC A61C 5/90

(57) ABSTRACT

A film tensioning element (10), with a lip ring (14) and a vestibular ring (16), between which, and possibly beyond which, a film (12) extends, wherein at least the vestibular ring (16) and/or the lip ring (14) is filled with a gas under pressure.

### CROSS-REFERENCE TO RELATED APPLICATIONS

This application claims priority to European Patent Application No. 20213108.2 filed on Dec. 10, 2020, the disclosure of which is incorporated herein by reference in its entirety.

### TECHNICAL FIELD

The invention relates to a film tensioning element.

### BACKGROUND

A film tensioning element of this type has been successfully used for more than ten years under the name OPTRAGATE to allow free access to the patient's mouth during dental work in the oral cavity of a patient. It consists of a vestibular ring and a lip ring with a film extending between them. In this element, the film is mounted so that it can slide relative to both rings and is elastic. It is latex-free in order to avoid allergic reactions.

A film tensioning element of this type is intended to cover areas to be protected inside the patient's mouth. This protects the dentist from infections on the one hand and the patient on the other. After application, an inserted film tensioning element must be disposed of. For this reason alone, it is important that it can be manufactured inexpensively and with low material costs.

In order to ensure access to the patient's mouth, both the lip ring and the vestibular ring of the film tensioning element consist of a comparatively stiff plastic material, with the current state of the art.

This ensures that the patient's mouth can be kept open to a sufficient extent. While the stiff design of a lip ring is usually not perceived as particularly unpleasant, patients complained that the hard vestibular ring of the first generation film tensioning elements was unpleasant for their gums and in the entire vestibular area.

In order to prevent rejection by the patients from getting out of hand, the vestibular ring was elaborately padded in the relevant areas by applying a softer plastic material in the second generation of film tensioning elements. This rather expensive solution is still being used, even though the material used is much greater and the production costs are also higher.

Such a film tensioning element can be seen in for example, EP 3 708 112 A1 and corresponding US2020188060A1 and US2021177251A1, which US published applications are hereby incorporated by reference in their entirety. Additional examples include US20170196659, US20180014914 and US20200188060, which US published applications are hereby incorporated by reference in their entirety.

For cost reasons, the version of the film tensioning element with a hard vestibular ring, which is perceived as uncomfortable, is often used.

### SUMMARY

In contrast, the invention is based on the task of creating a film tensioning element according to the claims, which offers excellent wearing comfort without increasing the manufacturing costs, while simultaneously, despite being made as a disposable part and so despite providing optimal protective properties against infections, the environment is less polluted.

This task is solved according to the invention by the independent claims. Advantageous further embodiments result from the sub-claims.

According to the invention, it is provided that the film tensioning element comprises two rings, namely a lip ring and a vestibular ring. The lip ring is intended to be arranged extraorally in the application of the film tensioning element, and the vestibular ring is intended to be arranged intraorally, namely in the vestibulum of the patient.

According to the invention, both rings are hollow. They are filled with a gas, in particular air, the gas being under pressure. This is deemed to mean that the internal pressure in the rings is at least as great as the ambient pressure, or greater.

Both rings are preferably designed as tubes and each tube forms a closed hollow space. According to the invention, it is advantageous for both rings to be made of the same material as the film that extends between the rings. The film may also extend beyond the vestibular ring and then be folded over at the vestibular ring.

The film and both rings are made of a highly elastic material, as is known per se. Preferably, the rings and film are integrated with each other. Examples of highly elastic materials useful herein, include but are not limited to, silicone, thermoplastic polyamidelastomers (TPA), such as PEBAX (from Arkema) or VESTAMID E (from Evonik_Industries), thermoplastic copolyesterelastomers (TPC) such as Hytrel (from Du Pont), Keyflex (from LG Chem) or Skypel (from SK Chemicals), thermoplastic elastomers based olefins (TPO) such as PP/EPDM, Elastron TPO or Saxomer TPE-O (from PCW), thermoplastic styrol block copolymers (TPS SBS, SEBS, SEPS, SEEPS and MBS) such as Elastron G, Elastron D, or Kraton (from Kraton Polymers), Septon (from Kuraray), Styroflex (from BASF), Thermolast (from Kraiburg TPE) ALLRUNA (from ALLOD Werkstoff GmbH & Co. KG) or Saxomer TPE-S (from PCW), thermoplastic elastomers based on urethane (TPU) such as Elastollan (from BASF), Desmopan, Texin or Utechllan (from Covestro), thermoplastic vulcanisates or cross-linked thermoplastic elastomers based on olefin, (PP/EPDM, TPV) such as Elastron V or Sarlink (from DSM) or Santoprene (from Exxon), or non classified thermoplastic elastomers of any composition or structure other than the above categories (TPZ).

It is preferable that the material is free from latex.

It is also possible to provide a compressed air channel that connects the cavities of the rings with each other. The rings together with the compressed air channel then form a single closed cavity.

One of the rings, preferably the lip ring, can be provided with a compressed air connection. This makes it possible to apply any desired pressure to this ring and preferably also to the vestibular ring.

As pressure increases each ring becomes stiffer and as pressure decreases each ring becomes softer and easier to deform.

In accordance with the invention, it is provided that both rings, when in use, have a stiffness such that the film can be stretched between them and the film tensioning element can be inserted into the patient's mouth with gentle manual squeezing of the vestibular ring into a somewhat oval shape.

In a relaxed state, both rings, when under pressure, preferably have a substantially circular shape.

Since both rings and the film are made in one piece, it is readily possible quickly to produce the entire film tensioning element using injection moulding. Random other production methods are also possible.

Typically, a dentist's treatment station has a source of compressed air. This source of compressed air can be operated by the dentist, for example by hand or foot, and can also be used to inflate the lip ring and the vestibular ring via an appropriate adapter. The stiffness of the rings can be adjusted by selecting the duration of the compressed air delivery. If the source of compressed air is in any case adjustable in terms of the pressure indicated and/or the air flow delivered, these settings options can also be used for inflating the lip ring and vestibular ring according to the invention.

Depending on the choice of their material thickness, the rings can have a shape that changes only slightly when inflated, so that the rings are simply fully inflated by the compressed air. However, it is also possible to expand the rings or at least one of the rings as if it were like a tube-shaped balloon by inflating it.

In this embodiment, the cross-sectional diameter of each ring can be increased, for example, threefold.

The effect of the compressed air on the rings depends to a large extent on the material thickness of the rings. For example, if the film has a material thickness of 0.3 mm, the rings can have the same material thickness. However, it is also possible to choose a material thickness of considerably more, for example 0.8 mm, for the rings. The possible range of material thicknesses of rings and film is between 0.05 mm and 1.2 mm, whereby in individual cases it is not impossible that material thicknesses above or below this range can be selected.

When inflated, the rings have a cross-sectional diameter that is significantly larger than when they are not inflated. Typically, the cross-sectional diameter is circular with a slight tip in the direction of the film. However, it is also possible to make the cross-sectional diameter circular or oval when the rings are inflated by selecting the appropriate injection mould.

For the production of the two rings, it is also possible to fold a tubular film with a diameter of 5 to 10 cm at either end, for example, over 8 mm.

The folded end is then welded onto the film, for example in an end area of 1 mm. Alternatively, bonding with an adhesive is possible.

Further random modifications of the invention are possible without leaving the scope of the invention. For example, it is possible to install a pressure reducer in the compressed air channel between the lip ring and the vestibular ring. This then has the effect that the vestibular ring is subjected to less excess pressure than the lip ring.

It is preferable that a film tensioning element is provided having a lip ring and a vestibular ring, between the lip ring and the vestibular ring, and optionally beyond which, a film extends, wherein at least the vestibular ring and/or the lip ring is filled with a gas under pressure.

It is preferable that both the lip ring and the vestibular ring are filled with gas, preferably air or ambient air.

It is preferable that the lip ring and/or the vestibular ring are inflatable.

It is preferable that the lip ring and/or the vestibular ring consists of a tube, wherein the tube is connected to the film extending between the rings.

It is preferable that the rings and the film are integral with each other and comprise the same material.

It is preferable that the lip ring has a compressed air connection via which it can be inflated.

It is preferable that the gas in both rings is in flow connection with one another via a compressed air channel.

It is preferable that a compressed air connection is or is configured to be connected to a source of compressed air having an adjustable source of compressed air, via which the vestibular ring and/or the lip ring are brought under an individually adjustable pressure.

It is preferable that both the lip ring and the vestibular ring as well as the film are fabricated of a highly elastic material and wherein, when the rings are inflated, both a diameter of the rings and a diameter of the film tensioning element increase.

It is preferable that a compressed air connection is provided, via which compressed air is releasable from the lip ring and/or vestibular ring, if required.

It is preferable that the rings are inflatable with an overpressure of 50 mbar to 200 mbar and that the rings when inflated increase their volume in comparison to a volume of the rings in a relaxed state, to a volume of at least three times the volume in the relaxed state.

It is preferable that the vestibular ring and/or the lip ring in the inflated state has or have a diameter which corresponds to at least one tenth of a distance between the rings when the film is stretched, and at most one third of the distance between the rings.

It is preferable that the compressed air connection is attached to the lip ring in a region of the lower lip, laterally to a patient's front teeth.

It is preferable that the material thickness of the rings is different, that the material thickness of the vestibular ring is greater than the material thickness of the lip ring.

It is preferable that the rings are formed during manufacture by folding over and fastening on or welding on, the film.

It is preferable that the vestibular ring is under a lower pressure or lower overpressure or under lower compression than the lip ring and wherein the vestibular ring comprises its own compressed air connection or a subsidiary compressed air connection or a pressure reducer.

It is preferable that a compressed air adapter is provided with which a compressed air connection of the film tensioning element can be connected to the compressed air outlet at a dental treatment station.

### BRIEF DESCRIPTION OF THE DRAWINGS

Further advantages, details and features are shown in the following description of several embodiments of the invention with reference to the drawings.

They show:

FIG. 1 a perspective view of an embodiment of a film tensioning element according to the invention;

FIG. 2 a rear view of the film tensioning element according to FIG. 1;

FIG. 3 a front view of the film tensioning element according to the invention in the embodiment according to FIGS. 1 and 2;

FIG. 4 a side view of the film tensioning element according to the invention in the embodiment according to FIGS. 1 and 2;

FIG. 5 a modified embodiment of the film tensioning element according to the invention;

FIG. 6 a view of a further embodiment of a film tensioning element according to the invention;

FIG. 7 a view of a further embodiment of a film tensioning element according to the invention;

FIG. 8 a view of a further embodiment of a film tensioning element according to the invention;

FIG. 9 a sectional view through the film tensioning element according to the invention, along line IX-IX, but in a modified embodiment; and

FIG. 10 a further sectional view through a further embodiment of a film tensioning element according to the invention.

### DETAILED DESCRIPTION

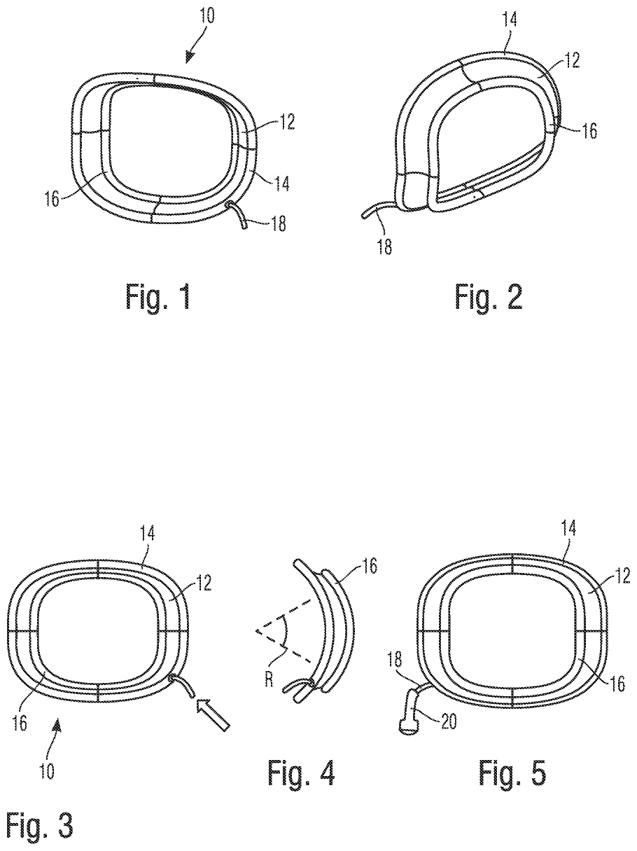

FIG. 1 shows a film tensioning element 10 in three-dimensional representation. The film tensioning element 10 is essentially circular in plan view. In the embodiment shown, the shape resembles a square with rounded corners.

However, any other shape, for example a circular shape or an oval or elliptical shape, is also possible instead.

It is advantageous if there is a certain approximation to the shape of a patient's mouth opening.

The film tensioning element 10 comprises a highly elastic film 12 extending between a lip ring 14 and a vestibular ring 16. Both rings 14 and 16 are in the form of tubes extending integrally from the film 12. The lip ring 14 and the vestibular ring 16 are filled with a pressurised gas. The gas may be an inert gas or, for example, ambient air.

Preferably, the rings 14 and 16 are inflatable. For this purpose, the film tensioning element 10 has at least one compressed air connection 18, where the compressed air connection 18 can be seen from FIG. 1.

Preferably, the compressed air port 18 comprises a check valve which prevents an inflated ring 14 from losing its air when the source of compressed air used for inflation is removed.

Only one compressed air port 18 is shown in FIG. 1 and the further figures. It goes without saying that provision must also be made for inflating the vestibular ring 16. For this purpose, the vestibular ring 16 also has a compressed air connection 18, which can extend, for example, starting from the vestibular ring 16 orally along the film 12, preferably laterally.

The film tensioning element 10 is shown from its front side in FIG. 1 and from its rear side in perspective in FIG. 2. The lip ring 14 is slightly larger than the vestibular ring 16 in a manner known per se. Preferably, the film 12 recedes slightly radially inwards in relation to the diameter of the two films, so that it has a lip-conforming structure.

For inserting the film tensioning element 10 into the patient's mouth, the lip ring 14 and the vestibular ring 16 are preferably inflated slightly, for example to a positive pressure of 100 mbar.

The film tensioning element 10 remains dimensionally stable and can be inserted into the patient's mouth. After insertion, the film tensioning element 10 is inflated to the desired overpressure, for example to 300 mbar, via the compressed air connection 18 and, if necessary, another compressed air connection.

In this state, spreading occurs and the dentist has good access into the patient's mouth, also, for example, to scan the vestibular side of the molars via an intraoral scanner.

The film tensioning element 10 is preferably manufactured in the same material thickness as the rings 14 and 16, and the two rings 14 and 16 are integral with the film 12. The material thickness can be adapted to the requirements in a wide range and is 0.2 mm in the illustrated embodiment example.

Due to the desired overpressure, each ring 14 and 16 expands relative to its limp and uninflated state, approximately to twice its cross-sectional diameter in the limp and uninflated state.

The lip ring 14 and the vestibular ring 16 are thus stiffened, i.e. by the air pressure, so that they can assume the desired substitute function for hard plastic rings, as they are provided in the prior art and can be avoided according to the invention.

FIG. 3 shows a plan view of a film tensioning element 10 in the embodiment according to FIGS. 1 and 2. It is shown that the film tensioning element 10 is symmetrical in itself and slightly wider than it is high, so that it is somewhat adapted to the shape of the mouth opening.

In FIG. 4, the film tensioning element 10 is shown in the inflated state of the rings 14 and 16. As shown, the entire film tensioning element 10 is curved about a radius R which is several centimetres outside and in front of the mouth opening.

The film 12 extends inwardly curved slightly radially to conform to the shape of the lips.

FIG. 5 shows a modified embodiment of a film tensioning element 10 according to the invention. The compressed air connection 18 is connected to a detachable compressed air adapter 20. This serves to use the compressed air available at the dental patient's treatment station to inflate the rings 14 and 16. Furthermore, in this solution, a not shown compressed air channel is provided, which extends between the rings 14 and 16.

Furthermore, in this embodiment, the cross-sectional diameter of the vestibular ring 16 is significantly larger than the cross-sectional diameter of the lip ring 14. This makes the vestibular ring 16 slightly softer and the lip ring slightly harder.

This distribution of stiffness can also be achieved by inflating the lip ring 14 to a greater overpressure than the vestibular ring 16. For example, a pressure reducer can be provided in the compressed air channel between the rings 14 and 16 to achieve this.

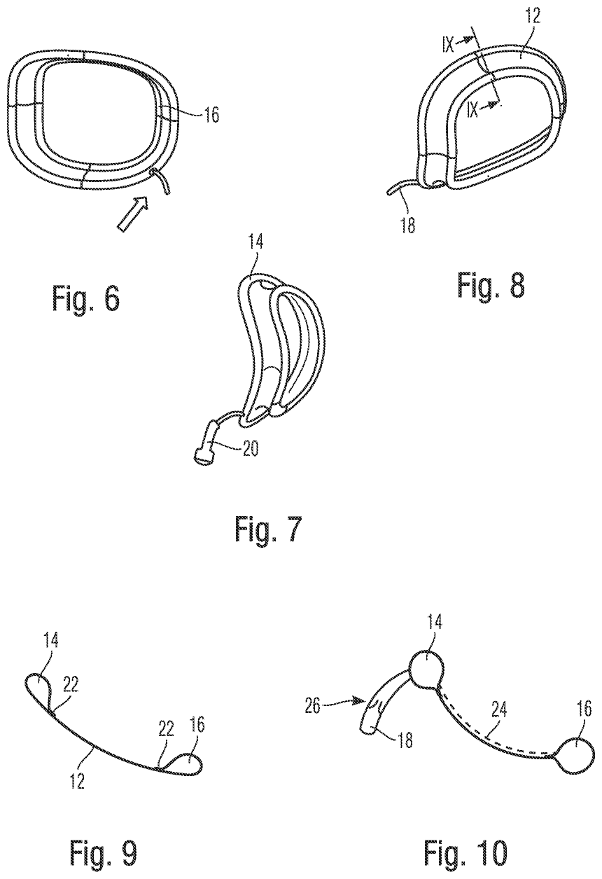

FIGS. 6, 7 and 8 show a further embodiment of the film tensioning element 10 according to the invention. The curvature of the film tensioning element 10 when inflated makes it particularly easy to insert the film tensioning element 10 into the patient's mouth.

The dentist uses his thumb and index finger to press the rings 14 and 16 together slightly in approximately the sagittal plane, so that the film tensioning element 10 becomes even wider, but less high. Then he first inserts one side of the distal end of the vestibular ring 16 into the patient's vestibule, then the mesial area, i.e., the area in front of the incisors, and after reducing the pressure between thumb and index finger the other distal area, which moves slightly mesially by reducing the pressure, so that insertion is made easier.

When the dentist completely releases the pressure on the lip ring 14 at the top and bottom, the film tensioning element 10 according to the invention automatically adapts to the shape of the patient's mouth, because the pressure exerted has a comparative effect and is therefore not perceived by the patient as annoying, in contrast to hard plastic rings which could produce pressure points and even injuries.

A section through the film tensioning element 10 according to the invention along line IX-IX is shown in FIG. 9. The course of the cut is indicated in FIG. 8.

In this embodiment, the film 12 is folded over at the end during manufacture, thereby forming the lip ring 14 and the vestibular ring 16. The folded end or its end region 22 is welded to the film 12 over a short distance by pressing the end there onto the film 12 while it cools.

In this embodiment, the lip ring 14 and the vestibular ring 16 are given a somewhat teardrop shape, as can be seen in FIG. 9.

It is understood that, as shown in FIGS. 1 to 8, at least one of the rings 14 and 16 has a compressed air connection 18 attached to it, and that each ring 14 and 16 forms a closed cavity when under pressure.

FIG. 10 shows a further embodiment. In FIG. 10, the way a compressed air channel 24 can extend between the rings 14 and 16 along the film 12 can be seen. The compressed air channel 24 may also be incorporated into the film 12 and likewise extend integrally therewith and along the rings 14 and 16.

The compressed air connection 18 can have the check valve 26 shown schematically in FIG. 10, which prevents the introduced compressed air from flowing out when the compressed air source is removed.

If it is necessary after all to release some compressed air, the dentist can compress the position of the non-return valve 26 in the compressed air channel 24 slightly between thumb and forefinger, so that the non-return valve 26 is no longer in operation and some air can be released.

The film tensioning element 10 according to the invention is particularly easy and inexpensive to manufacture, since it consists exclusively of the highly elastic film 12, which can also be used with a constant film thickness.

1. A film tensioning element comprising
a lip ring and
a vestibular ring,
between the lip ring and the vestibular ring, and optionally beyond which, a film extends,
wherein at least the vestibular ring (16) and/or the lip ring (14) is filled with a gas under pressure.

2. The film tensioning element according to claim 1,
wherein both the lip ring (14) and the vestibular ring (16) are filled with gas.

3. The film tensioning element according to claim 1,
wherein the gas is air or ambient air.

4. The film tensioning element according to claim 1,
wherein the lip ring (14) and/or the vestibular ring (16) are inflatable.

5. The film tensioning element according to claim 1,
wherein the lip ring (14) and/or the vestibular ring (16) consists of a tube, wherein the tube is connected to the film (12) extending between the rings (14,16).

6. The film tensioning element according to claim 1,
wherein the rings (14, 16) and the film (12) are integral with each other and comprise the same material.

7. The film tensioning element according to claim 1,
wherein the lip ring (14) has a compressed air connection (18) via which it can be inflated.

8. The film tensioning element according to claim 1,
wherein the gas in both rings (14, 16) is in flow connection with one another via a compressed air channel (24).

9. The film tensioning element according to claim 1,
wherein a compressed air connection (18) is or is configured to be connected to a source of compressed air comprising an adjustable source of compressed air, via which the vestibular ring (16) and/or the lip ring (14) are brought under an individually adjustable pressure.

10. The film tensioning element according to claim 1,
wherein both the lip ring (14) and the vestibular ring (16) as well as the film (12) comprise a highly elastic material and
wherein, when the rings (14, 16) are inflated, both a diameter of the rings and a diameter of the film tensioning element (10) increase.

11. The film tensioning element according to claim 1,
wherein a compressed air connection (18) is provided, via which compressed air is releasable from the lip ring (14) and/or vestibular ring (16), if required.

12. The film tensioning element according to claim 1,
wherein the rings (14, 16) are inflatable with an overpressure of 50 mbar to 200 mbar and
wherein the rings when inflated increase their volume in comparison to a volume of the rings in a relaxed state, to a volume of at least three times the volume in the relaxed state.

13. The film tensioning element according to claim 1,
wherein the vestibular ring (16) and/or the lip ring (14) in the inflated state has or have a diameter which corresponds to at least one tenth of a distance between the rings (14, 16) when the film (12) is stretched, and at most one third of the distance between the rings (14, 16).

14. The film tensioning element according to claim 1,
wherein the compressed air connection (18) is attached to the lip ring (14) in a region of the lower lip, laterally to a patient's front teeth.

15. The film tensioning element according to claim 1,
wherein the material thickness of the rings (14, 16) is different, the material thickness of the vestibular ring (16) is greater than the material thickness of the lip ring (14).

16. The film tensioning element according to claim 1,
wherein the rings (14, 16) are formed during manufacture by folding over and fastening on or welding on, the film (12).

17. The film tensioning element according to claim 1,
wherein the vestibular ring (16) is under a lower overpressure than the lip ring (14) and
wherein the vestibular ring (16) comprises its own compressed air connection (18) or a subsidiary compressed air connection or a pressure reducer.

18. The film tensioning element according to claim 1,
wherein a compressed air adapter (20) is provided with which a compressed air connection (18) of the film tensioning element (10) can be connected to the compressed air outlet at a dental treatment station.
